(19) United States
(12) Patent Application Publication
(10) Pub. No.: US 2014/0088440 A1
(43) Pub. Date: Mar. 27, 2014

(54) Stereoscopic Plug-And-Play Dermatoscope And Web Interface

(71) Applicants: Elliot Krishna Swart, Palo Alto, CA (US); Elizabeth Mary Asai, Leesburg, VA (US); Nickolas Peter Demas, Manlius, NY (US)

(72) Inventors: Elliot Krishna Swart, Palo Alto, CA (US); Elizabeth Mary Asai, Leesburg, VA (US); Nickolas Peter Demas, Manlius, NY (US)

(21) Appl. No.: 14/119,301

(22) PCT Filed: May 25, 2012
(86) PCT No.: PCT/US2012/039546

§ 371 Date: Nov. 21, 2013

Related U.S. Application Data
(60) Provisional application No. 61/490,178, filed on May 26, 2011.

(51) Int. Cl. A61B 5/00; A61B 5/107

(52) U.S. Cl. CPC A61B 5/0077; A61B 5/444; A61B 5/1079; A61B 5/0013; A61B 5/0022; A61B 5/1077; A61B 5/742; A61B 2576/02
USPC 600/476

(57) ABSTRACT

A system includes a device (100) for imaging a skin abnormality of a patient, and a web interface (115, 125). The imaging device has a camera (103) recording a plurality of images from locations separated by that distance, thereby obtaining a stereoscopic image of the skin abnormality. The imaging device may be configured as a handheld unit, with the camera a plug-and-play webcam and the device having a USB connection (114) to a computer (110, 120). The web interface (115, 125) links the computer (110, 120) to a web site providing access to storage of the image data. The web interface provides a patient portal for entering information regarding the skin abnormality, and a doctor portal for accessing patient medical history and entering clinical data regarding the skin abnormality. The system may further include a server (160) to receive and analyze the image data, generate 3D stereoscopic images of the skin abnormality, and compute metrics for clinical evaluation of the skin abnormality.

### FIELD OF THE DISCLOSURE

The present disclosure relates to medical imaging, and more particularly to an imaging device and web interface for recording and monitoring skin abnormalities, including potential melanomas.

### BACKGROUND OF THE DISCLOSURE

Two million Americans develop skin cancer every year, meaning that one in five will be diagnosed in their lifetime. Of these incidents, 60,000 are melanoma, the deadliest form. Melanoma alone is responsible for 8,000 patient deaths each year. Out of all skin melanomas, 30% begin as moles and 90% of all moles contain carcinogenic mutations. Skin cancer is most deadly if not detected in its early stages. Improved mole screening will facilitate diagnosis of melanoma in earlier stages, leading to a better prognosis and likely lowered cost associated with treatment.

Accordingly, there is a need for a system that can monitor potentially carcinogenic skin lesions, and detect cancerous activity in the earliest, most treatable stages. Patients with malignant lesions may then be diagnosed at earlier stages of their disease (thus decreasing the cost of their treatment), while patients with benign lesions may be able to have their lesions inspected from home by a specialist, reducing the number of unnecessary doctor's visits.

Furthermore, it is desirable for patients to feel comfortable when using this device while logging images of skin abnormalities. In particular, it is desirable to provide an imaging device that is easy to use and inexpensive enough so that patients may be able to take standardized skin scans in the comfort of their homes. It also is desirable to provide physicians with consistent, standardized lesion information obtained between regular appointments. Typically, changes in an abnormality between visits are only brought to a physician's attention if the patient notices the change and determines it warrants another appointment. It is desirable to implement a system for tracking such changes on a more frequent, regular schedule, with software to evaluate new images and report significant changes to physicians to permit fast response.

### SUMMARY OF THE DISCLOSURE

The present disclosure addresses the above-described need by providing a system, including a dermatoscope and a web interface, for obtaining standardized, high definition photo records of skin abnormalities at a price low enough for widespread use in clinics and homes. The system's web interface tracks these lesions over time with 2D and 3D images, and performs analysis that highlights significant changes in the abnormality. These images and changes are logged into a historical record on a secure web database. This interface allows doctors access to patient information safely in any location with internet access.

In accordance with an embodiment of the disclosure, the dermatoscope has 3D imaging capability to provide physicians with accurate and realistic images that show height and depth of skin abnormalities, thus permitting quantitative elevation measurements. This represents a significant improvement over qualitative measurements of elevation and volume typically made by physicians; imaging the third dimension enables doctors to visually identify more changes between sequential images.

According to an aspect of the disclosure, the system is easy enough to use so that patients feel comfortable when using this device while logging individual images. Patients are able to log in to a personalized “Patient Portal” and view a simple, user-friendly listing of all previous scans. By making these entries, the patient automatically creates a documented history of each abnormality. Patients are thus encouraged to consistently look for suspicious lesions and immediately bring them to the attention of a physician instead of waiting to schedule an appointment.

According to another aspect of the disclosure, the system provides physicians with consistent, standardized lesion information obtained between regular appointments. The system is suitable for monitoring skin abnormalities from the patient's home, as well as to alert the doctor to suspicious skin activity between appointments. The system allows for observed changes in a patient's skin lesion to be tracked on a more frequent, regular schedule. In an embodiment, automatic online imaging software evaluates new images and reports significant changes to physicians to allow for fast response. The system includes an interface with a “Doctor Portal” allowing a physician to view a patient's home progress, check flagged changes and consult other doctors on troubling abnormalities. In an embodiment, the system allows for the creation of an accurate patient history, and allows that history to be seamlessly utilized by both primary care physicians and dermatologists to improve early detection of life threatening conditions. The dermatoscope works with a fully integrated web interface to automatically setup and record a dermatological scan as well as make lesion history accessible to both patient and doctor.

According to an aspect of the disclosure, a system includes an imaging device for imaging a skin abnormality of a patient, and a web interface. The imaging device includes a camera and a controller. The controller controls linear motion of the camera over a predetermined distance so that the camera records a plurality of images from locations separated by that distance, thereby obtaining a stereoscopic image of the skin abnormality. The system also includes a data path for transmission of image data to a computing device. The web interface links the computing device to a web site providing access to storage of the image data. In an embodiment, the camera is mounted on a track, and a servo motor, connected to the camera and to the controller, causes linear motion along the track. The imaging device may advantageously be configured as a handheld unit, with the camera and data path being respectively a plug-and-play webcam and a USB connection. In an embodiment, the web interface provides a patient portal for entering patient information and information regarding the patient's skin abnormality, and a doctor portal for accessing patient medical history and for entering clinical data regarding the patient's skin abnormality. The system may further include a server configured to receive and analyze the image data, generate 3D stereoscopic images of the skin abnormality, and compute metrics for clinical evaluation of the skin abnormality.

### BRIEF DESCRIPTION OF THE DRAWINGS

FIG. 1 schematically illustrates a system including a dermatoscope and web interfaces in accordance with an embodiment of the disclosure.

FIG. 2 schematically illustrates a dermatoscope according to an embodiment of the disclosure.

FIG. 3 shows a cross-polarized image of a skin lesion obtained from an imaging system according to an embodiment of the disclosure.

FIG. 4 is a screenshot of a patient portal displaying data entries regarding skin abnormalities, according to an embodiment of the disclosure.

FIG. 5 is a screenshot of an update of a skin abnormality using the patient portal of FIG. 4.

FIG. 6 is a screenshot of a doctor portal displaying an image of a skin abnormality and properties of the image, according to an embodiment of the disclosure.

FIG. 7 is a chart illustrating information security in a system embodying the disclosure.

FIGS. 8A, 8B and 8C respectively illustrate a skin abnormality image, a thresholded image of the abnormality, and an image of the abnormality with a drawn boundary provided by a system embodying the disclosure.

FIGS. 9A and 9B respectively illustrate a skin abnormality image and a height map provided by a system embodying the disclosure.

FIG. 10 shows a handheld imaging device according to an embodiment of the disclosure.

### DETAILED DESCRIPTION

A system embodying the present disclosure, referred to herein as “3Derm,” is described in more detail below.

### System Overview

The 3Derm system consists of two main components: a handheld, stereoscopic dermatoscope and a web interface for patients and physicians. In this embodiment, stereoscopic imaging is obtained by using a webcam to record two images along an axis to mimic viewpoints of the left and right eyes. The dermatoscope is plug-and-play, allowing any user with Internet access to connect the device to a computer via USB and use the web interface. Once connected, the interface provides user-friendly instructions to help a patient navigate the “Patient Portal”, take image scans and navigate through his or her scan history. Physicians have a similar interface, the “Doctor Portal”, which allows doctors to easily select a patient, review past and current stereoscopic images of each skin abnormality and view metrics characterizing the abnormality's change over time.

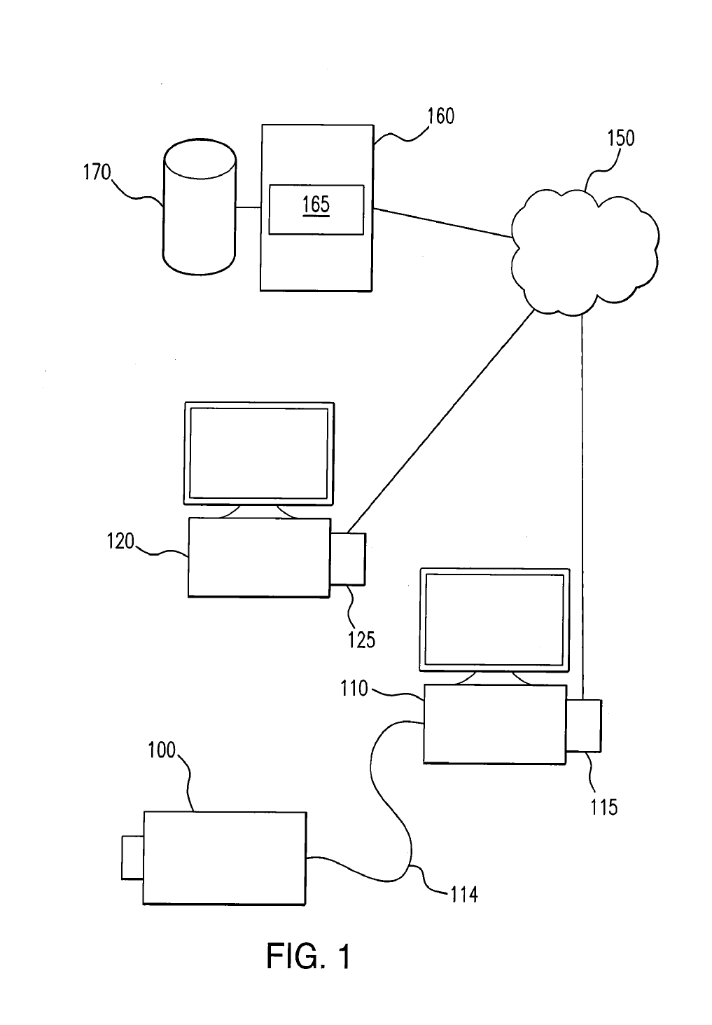

FIG. 1 schematically illustrates a 3Derm system according to an embodiment of the disclosure. A handheld dermatoscope 100, capable of 3D imaging, is connected to computer 110 by a USB connection 114. In this embodiment, the dermatoscope is used in the patient's home, and the patient's computer 110 has a web interface 115 including a “Patient Portal,” described in more detail below. A physician's computer 120, typically located remote from the patient, has a web interface 125 including a “Doctor Portal.” The computers are linked via a network 150 such as the Internet. Software 165 for managing the system is located on server 160, which is also connected to the network and typically is remote from computers 110, 120. A storage device 170 is linked to the server and has a database of patient information, including patient histories and data regarding patients' skin abnormalities. Software 165 supports a web site that is accessed by a user (typically a patient or physician) via interface 115, 125. In an embodiment, all patient data, image data, and image analysis results are stored on the remote device 170. A user is not required to install any of the 3Derm system software, but instead accesses the website via the web interface.

### Dermatoscope

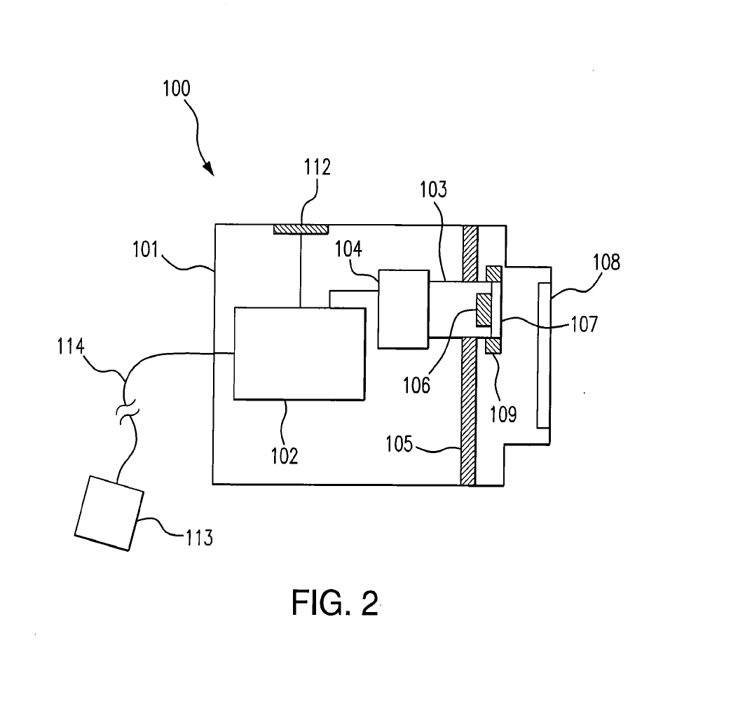

FIG. 2 shows details of 3D imaging dermatoscope 100 according to an embodiment of the disclosure. Control board 102 is connected to micro-servo motor 104 which moves webcam 103 along a track 105. Webcam 103 includes a camera sensor 106; a polarized filter 107 is mounted in front of the camera sensor. The webcam has integrated illumination; LED lights 109 surround the sensor 106 and filter 107. Control board 102 sends image data to computer 110 via USB cable 114; USB connector 113 plugs into computer 110. The control board, servo, webcam and camera track are enclosed in a plastic shell 101. The mechanism and components are integrated into a unibody design, decreasing the number of parts and increasing ease of assembly, calibration and device durability. The dermatoscope is designed to be held comfortably in the user's hand.

A touch sensor 112 is located on top of the device. Touch sensor 112 is connected to control board 102; touching the sensor initiates the imaging sequence. The micro-servo 104 is actuated to produce the webcam's linear motion. During image capturing, the dermatoscope takes one image, and then translates the camera laterally to a position 3 mm away to capture a second image. In order to ensure that the device has not been moved during the process, the camera is returned to the original position to take a third image. If the first and third images do not match, the user is instructed to repeat the scan.

Webcam 103 may be moved by a variety of alternate mechanisms and methods. For example, a linear actuator may be used instead of a servo.

The two 2D images are combined into one 3D stereoscopic image. This stereoscopic method of obtaining 3D images is advantageous because it requires only 3 still images of the abnormality, has a total capture sequence time of less than 6 seconds and uses LED lights for illumination. Stereoscopic images obtained from these viewpoints may then be visualized on a 3D color display.

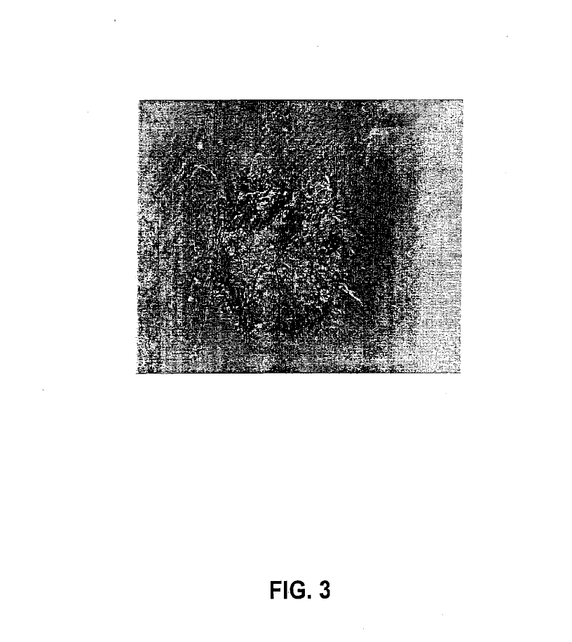

When taking pictures of skin illuminated with a non-polarized source, the skin reflects much of the incident light. These reflections tend to obscure surface details. To address this problem, cross-polarized illumination is used. A polarized filter 108 is inserted in front of the LED illumination, with polarization orthogonal to that of filter 107. This arrangement permits capture of reflection-free images and provides sufficient contrast to visualize underlying lesion structure otherwise not visible (see FIG. 3).

In this embodiment, the dermatoscope incorporates a plug-and-play webcam with integrated illumination. A user may connect the dermatoscope via USB to any available personal computer, log in to the web interface and take the first scan within minutes. The webcam requires no user calibration.

Any 3D-capable computer may be used to visualize the 3D images (for example, a Sony Vaio® F Series 3D laptop). Both patients and doctors can also view the results of a scan in 2D on a standard monitor. Portable, handheld devices may also be used to visualize 3D images; for example, the Nintendo 3DS® where one can visualize a stereoscopic image without specialized eyeglasses.

The procedure for performing a scan process is detailed in a user-friendly format on the web interface. Patients are not required to email files to doctors or for the doctors to store and catalog a large volume of images. Dermatological images, obtained in the patient's home, are logged in online database storage. As the process for capturing stereoscopic images is mechanically uncomplicated, image capture may be improved using software side updates.

### Web Interface

The web interface 115, 125 provides a comprehensive system for patients and their doctors to monitor skin irregularities over time. The interface has both a Patient Portal and Doctor Portal to allow patients, doctors and dermatological specialists to input and access information.

Using a driverless (plug-and-play) webcam permits convenient operation of the system. In this embodiment, the webcam follows the USB Video Class driverless specification, so that it is fully compatible with various operating systems (e.g., Microsoft Windows XP®, Intel Mac, and others). Because the dermatoscope is driverless and the software is web-based, participating doctors may simply navigate to the website and connect the imager. This setup time is generally significantly less than if the 3Derm system required a full installation on each machine.

Because the imager is implemented as a webcam, the client software must rely on analyzing the video feed to determine when images should be captured. The client software includes a motion sensing algorithm for monitoring the video feed and waits for the image to be still for 2 seconds. The interface then prompts the user to initiate the imaging sequence. When the touch sensor is actuated, the imager's microcontroller directs the micro-servo to move and stop the camera at pre-programmed times. Responding to that motion, the software starts a timer that allows for the left and right images to be captured at the correct stereoscopic viewpoints.

A cloud-based data storage approach offers several advantages over traditional, on-site storage. Doctors would not be required to save information on their file systems, because the database will be secured to the standards of the Health Insurance Portability and Accountability Act (HIPAA) and backed up with HIPAA approved services. By storing the data on the server, patients and doctors can access their files from any computer with Internet access. For doctors who want on-site storage, data from the server could be regularly downloaded and integrated into their file system.

Because the software is web-based and requires no installations, updates would be made without version compatibility issues or required patches. In this embodiment, Microsoft Silverlight® is installed on user computers as a Rich Internet Application (RIA) client side architecture used to build the web interface. Client side architecture is a web building approach that allows for the computationally intensive work to be downloaded to the user's computer.

### Patient Portal

The interface for patients is designed to be simple and intuitive. In an embodiment, a new user navigating to the website is presented with a welcome screen where the user (typically a patient) can create a new account or input login information; if creating a new account, the patient is asked to complete a survey regarding their medical history. The interface also has a field to input the name of the patient's current doctor, so that the doctor may access their patient's information.

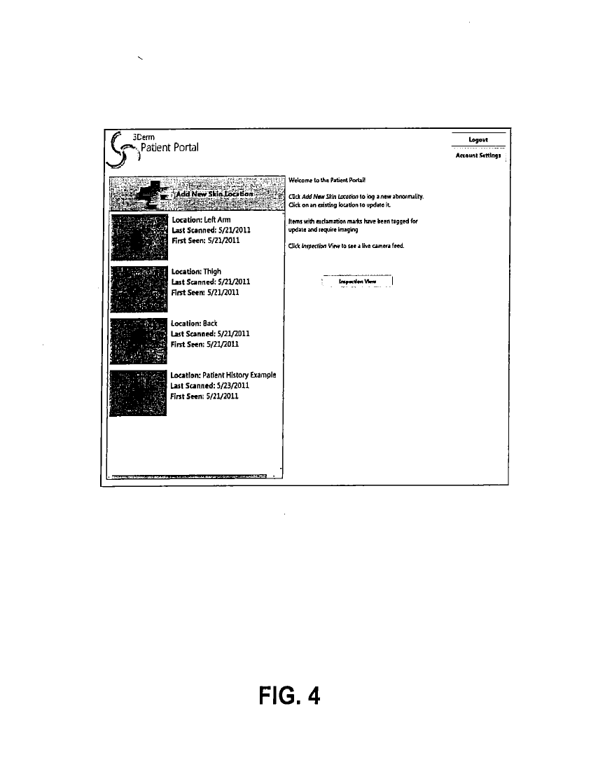

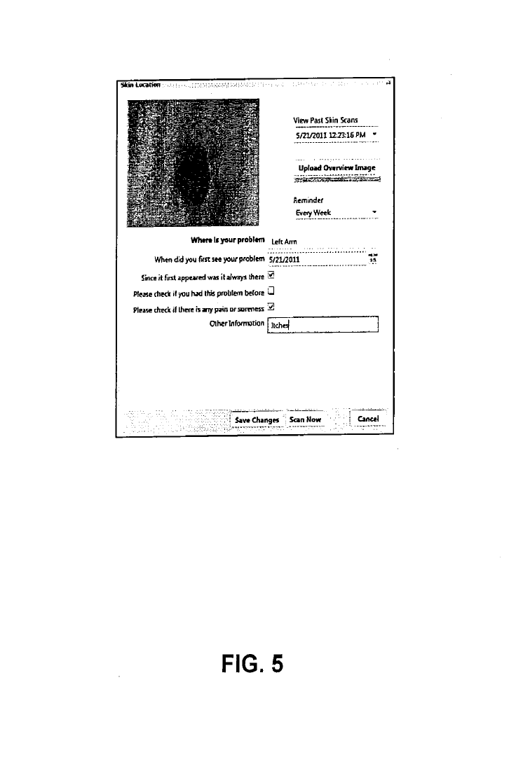

After creating an account and/or logging into the Patient Portal, the patient can view a list of all previous skin abnormalities (see FIG. 4). Each entry can be expanded to see past images, and to update past skin abnormalities (see FIG. 5). Doctors or patients can set how frequently the abnormality should be scanned; in an embodiment, the server is designed to send out email reminders according to this schedule. This will help patients remember to take regular skin scans.

When the patient logs on, abnormalities that require imaging appear at the top of the list with an exclamation mark. If a patient finds a new abnormality but for various reasons cannot make an appointment, the new skin abnormality can be imaged and sent to his or her doctor for inspection. All uploaded scans from the Patient Portal are automatically updated in the corresponding Doctor Portal. This allows for seamless and secure transfers of medical information, avoiding the need for chains of emails and attachments between patients and their physicians.

In addition to performing skin scans with the imager, a patient may use the Patient Portal to stream a live 2D video feed to the screen. This permits patients to visualize locations on their body that are otherwise hard to see and find lesions that they may not have noticed otherwise. Patients using this option will potentially find more lesions, take more image scans and detect more melanomas in their earliest stages.

While the 3Derm system is designed to view skin on a small scale, having the capability to get an overview of the surrounding skin is diagnostically important in some cases. In these situations, the patient may take an overview image using a digital camera and easily upload and pair it with the lesion, giving a doctor two perspectives of an abnormality.

### Doctor Portal

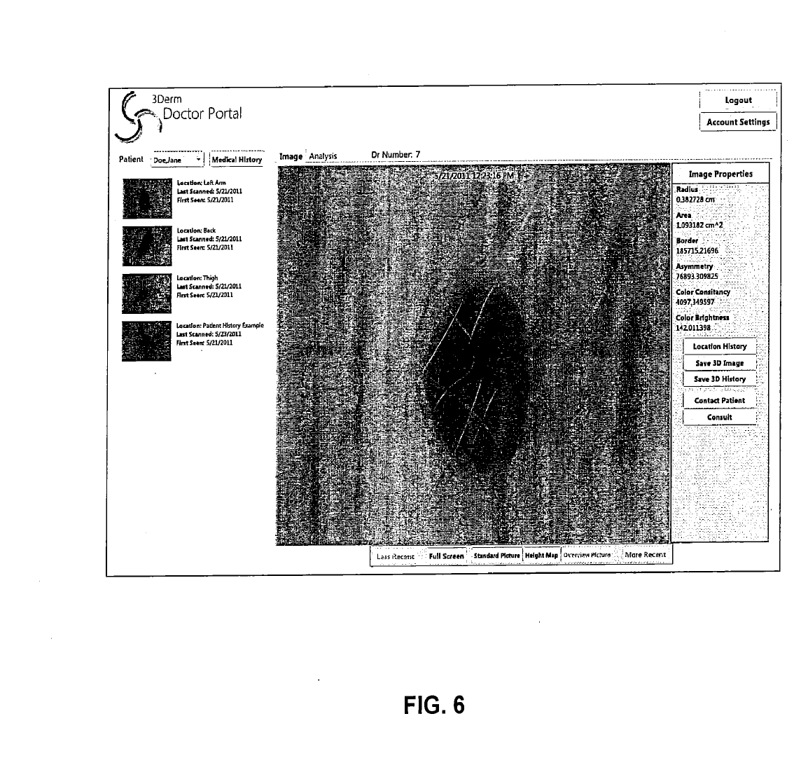

Doctors use the same website as the patients. After logging in, they are redirected to the Doctor Portal (FIG. 6). This interface was designed to allow physicians easy access to the recorded images and analysis. The Doctor Portal has a drop down menu for choosing a particular patient and a list similar to that of the Patient Portal detailing each scanned abnormality. The physician may view these images and compare previous scans of the same lesion. Any time a patient scans a new lesion, the corresponding physician's Doctor Portal will be updated with the new information.

The server's image analysis data is displayed in a separate tab of the Doctor Portal. The different parameters of a specific lesion including radius, area, border, asymmetry and color data are displayed by each entry. Detailed graphs display how these variables have changed over time. A physician can also toggle between the image and a height map, which displays elevation. If a significant change is detected, the entry will be flagged as having suspicious activity.

Sudden changes in a skin lesion require immediate attention. For this reason, the interface gives physicians the capability to immediately notify a patient of a suspicious change. The doctor can either use the contact phone number displayed, or for a less urgent follow up, send a notification email to the patient's inbox. The system accordingly offers seamless notifications and communication seamless.

The interface uses a de-identified file sharing system to allow doctors to easily consult other physicians on interesting or strange lesions. For example, if a primary care physician encounters a lesion that might warrant a patient referral, the physician could image the skin abnormality and share the data with a consulting dermatologist. This system of collaboration decreases unnecessary referrals without compromising patient health information or HIPAA regulations. Specialists may also use this channel of communication to update the referring doctor on any abnormality's status.

### Skin Color Settings

In the field of dermoscopy, imaging devices must take patient skin color into account because the analysis and diagnoses are based on identifying specific color pigments. For this reason, the web interface has a configurable setting for skin tone. A patient may select between light, medium or dark skin in order to obtain the best quality images and analysis. These settings were designed to increase the population of potential users. The patient may also select for low, medium, or high volumes of hair in the imaging region. In another embodiment, this process may be done automatically.

### Information Security

HIPAA sets strict guidelines on safeguarding data when dealing with personal health information (PHI). In an embodiment, the interface uses Microsoft Server 2008 R2 and Microsoft Structured Query Language (SQL) Server 2008 R2 for the server and database respectively, as is used by numerous HIPAA secured hosting companies. This architecture permits HIPAA compliance with minimal difficulty.

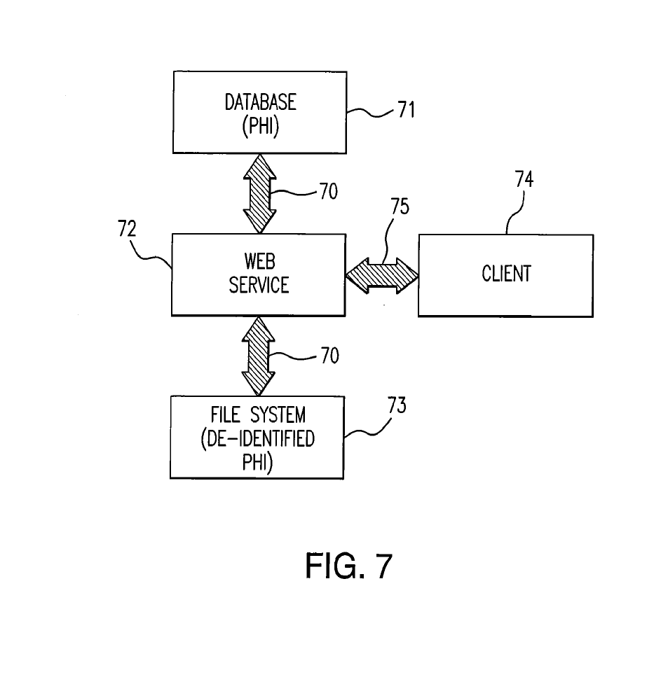

As shown in FIG. 7, database 71 storing PHI, web service 72 and file system 73 (which may include de-identified PHI) are maintained on the server. The web application and web services are designed to be HIPAA compliant. This means that only patients and their doctors can view PHI. In this embodiment, Windows native authentication 70 is used between the web services and the database in order to check authentication on every request. This system blocks malicious client programs, as well as any other malicious users, from gaining access to data without the proper email address and password. A strict, cross-domain policy is employed so that no application running on other websites can access the web services, insuring the security of patient data. At no point in time does the web service 72 release any identified patient data passed to any client without the proper authorization. Access to the database is regulated through the Microsoft LINQ middleware, which is designed to intercept and eliminate any SQL injection attacks, further securing the server. In addition, SSL encryption 75 prevents the interception of data moving between the client 74 and the server.

The database 71 (in this embodiment, a Microsoft SQL database) is also designed to be HIPAA compliant. The database strictly limits user access, has total encryption, logs all changes, and allows for emergency retrieval of data. These tasks adhere to basic HIPAA guidelines for electronic storage.

A system according to an embodiment of the disclosure allows for a large database of two and three-dimensional de-identified images to be collected from consenting patients. This can not only vastly expand current medical image libraries but also help train doctors to diagnose skin conditions from clinical images. Furthermore, this de-identified information may be continuously uploaded in real-time; ongoing studies therefore may use the image data without requiring an additional time commitment from the patient.

### Clinical Study

A clinical study was performed in order to test the usability, functionality and accuracy of the 3Derm system in identifying suspicious lesions. Patients' skin abnormalities were imaged using a handheld dermatoscope by an on-site doctor viewing the abnormalities in person. The dermatoscope was connected to a computer via USB; the images were automatically uploaded to the server and saved in the database. The on-site doctor also recorded a preliminary diagnosis and whether or not a biopsy was ordered.

The 2D and 3D images, as well as the location of the abnormality, were then shown to a panel of dermatologists, who had not seen the abnormality in person. The panel doctors then noted remotely a preliminary diagnosis and if a biopsy would need to be ordered. The panel doctors' decisions were then compared to that made by the on-site doctor. If a biopsy had been performed, all responses were put in the context of the actual histological results.

In comparing the on-site doctors' decisions to those of the panel, three factors were examined: the doctors' agreement on whether to biopsy, their preliminary diagnosis, and biopsy results. The decision to conduct a biopsy was considered the most important parameter.

A total of 52 abnormalities were imaged. Five images were excluded due to doctor input and upload error, leaving 47 scans for review. Two panel doctors viewed each abnormality and thus 94 biopsy and diagnosis results were recorded. The results of the clinical study, shown below in Table 1, are categorized to reveal and compare the doctors' decisions to biopsy the lesions and their preliminary diagnoses.

TABLE 1
Relationship between Panel Doctors' and
On-Site Doctor's Decision to Biopsy

| | | Percentage |
|---|---|---|
| Comparison of Biopsy Orders | Number of Trials | of Total Seen |
| Agreements | 70 | 74.5 |
| On-Site No Biopsy, Panel Biopsy | 19 | 20.2 |
| On-Site Biopsy, Panel No Biopsy | 5 | 5.3 |
| Total Reviewed | 94 | 100 |

Agreements are defined as instances where both on-site and remote doctors ordered or did not order a biopsy, regardless of the biopsy result.
On-Site No Biopsy, Panel Biopsy referred to trials where the panel doctors would have ordered a biopsy when the on-site doctors did not order a biopsy.
On-Site Biopsy, Panel No Biopsy referred to trials where the panel doctors would not have ordered a biopsy when the on-site doctors did order a biopsy.

The results indicate that doctors who viewed scanned images remotely were reliably able to determine which abnormalities warranted a biopsy. The panel and on-site dermatologists were in full agreement on 74.5% of the abnormalities. This means that these lesions were given the same biopsy decision when seen in person or in image form. All trials with positive cancerous results were in this category, as every dermatologist agreed to biopsy the cancerous lesions.

Table 2 compares cancerous results, false positives, and false negatives in the set of biopsies ordered by the on-site doctors and the panel doctors. The assumption is made that any lesion not biopsied by the on-site doctor was benign.

TABLE 2
Number of False Positives and False Negatives Found
for Biopsied Lesions

| | On-Site Doctors | | Panel Doctors | |
|---|---|---|---|---|
| Cancerous Results | 5 | (27.8%) | 10 | (18.5%) |
| False Positives | 13 | (72.2%) | 44 | (81.5%) |
| False Negatives | 0 | (0%) | 0 | (0%) |
| Biopsies Ordered | 18 | (100%) | 54 | (100%) |

Cancerous Results refer to trials where the biopsy samples ordered tested positive for at least one type of cancer
False Positives are defined as trials where the biopsy result was negative (non-cancerous) but the doctors ordered biopsies
False Negatives are defined as trials where the biopsy result was positive (cancerous) but the doctor did not order a biopsy
Biopsies Ordered refers to the total number of biopsies indicated by the specialist. All on-site biopsy orders (18) were biopsied, while Panel Doctors' biopsy orders (54) were lesions that would have been biopsied

Doctors biopsy a significant number of lesions that are considered suspicious, but not cancerous. Of the 18 lesions biopsied by the on-site dermatologists, only 28% were cancerous. This means that in a normal clinical setting, 72.2% of the biopsied lesions could be considered false positives. The results showed that in remote diagnosis, the rate of panel false positives increased only to 81.5%. This 9.3% increase is reasonable as doctors looking only at images would be more cautious and likely biopsy a suspicious abnormality. The panel doctors had no false negatives, meaning that no known cancerous lesion was left un-biopsied.

While these results indicate a greater number of biopsies ordered by the panel physicians viewing the image evidence alone, the overall number of patient trips to the clinic would be decreased. Patients with lesions that were obviously benign would not need to come into the doctor's office. It is presumed that the panel doctor would personally see a patient who had lesions determined remotely to require a biopsy. The lesion, if unsuspicious, would be determined to not require biopsy during this visit. These results show that specialists remotely looking at the images can identify which lesions require attention with zero false negatives and only a slight increase in the number of false positives. This would make the system practical as a monitoring tool that would allow a specialist to remotely determine when the physical presence of the patient was needed for biopsy.

The diagnosis component of the panel review assessed the system's ability to diagnose remotely. In order to standardize the study, each panel doctor was only given the location and images of the abnormality. Table 3 shows a comparison of the on-site doctor's and panel doctors' diagnoses.

TABLE 3
Relationship Between On-Site Doctor's and Panel Doctors' Diagnoses

| Comparison of Diagnoses | Number of Trials | Percentage of Total Seen |
|---|---|---|
| Agreements | 56 | 59.6 |
| Visually Identical | 5 | 5.3 |
| Seborrheic Keratosis vs. | 6 | 6.4 |
| Benign Nevus | | |
| Different Skin Cancers | 8 | 8.5 |
| Disagreements | 19 | 20.2 |
| Total | 94 | 100 |

Agreements are defined as trials where the on-site and panel doctors recorded the same preliminary diagnosis.
Visually Identical trials are defined as those involving skin conditions that are so similar that they usually require a biopsy to differentiate between diagnoses. All conditions that would present in the same visual manner but were confused for each other were put into this category.
Distinguishing between lesions such as lentigo and macular seborrheic keratosis or lichenoid keratosis and basal cell carcinoma can be very difficult even if on-site.
Seborrheic Keratosis vs. Benign Nevus trials are defined as those that confused a seborrheic keratosis with a benign nevus, or vice versa. To differentiate between these two conditions, a dermatologist must determine if the surface of the abnormality is smooth or scaly. The results indicated that in some cases, the present image quality does not display the required clarity for differentiation. However, both of these conditions are benign with neither requiring a biopsy.
Different Skin Cancers are trials involving skin abnormalities diagnosed as two different families of skin cancers. The inventors found it significant to highlight those trials in which a dermatologist could at least identify the lesion as a cancerous abnormality.
Disagreements are identified as trials in which the panel disagreed with the original physician's preliminary diagnosis. In those trials, the two diagnoses were different beyond a factor of similar appearances, or same family of disease. Improving the device's image quality is targeted at reducing this number.

Several factors must be taken into account when examining these results. As with all visible light imagers, the imager faces the problem of differentiating between skin abnormalities with highly similar appearances. On some lesions, in-person or remote inspection will only narrow the doctor's preliminary diagnosis between two possibilities without biopsy results. Other lesions are similar in appearance, but require the texture properties of the lesion in order to make an accurate diagnosis. Panel doctors were only given locations and images of the lesions. Panel doctors may have been able to better assess a lesion if the medical history of the patient had been provided. In addition, the doctors involved in this study had varying degrees of experience with clinical photos; it is known that diagnostic accuracy is correlated with years of experience and familiarity with photo-diagnosis.

While the dermatoscope can obtain images and compute lesion characteristics, the system is not meant to replace the role of dermatologists. Based on the preliminary data, the current prototype should not be used as the sole basis to diagnose a patient or to determine a course of treatment for a skin abnormality. However, after accounting for the inherent diagnostic difficulties posed by limited patient information and the inexact nature of visual examination, the 3Derm system was shown to give panel doctors a number of diagnostically useful images.

According to other studies of teledermatology, an acceptable level of reliability for teleconsultation was determined to be 60% or higher. In the study describe above, panel doctors were in agreement with the on-site doctor 59.6% of the time. Accounting for the additional 5.3% associated with visually identical diagnoses would bring the system's accuracy rate to 64.9%, as both the on-site and panel doctor's preliminary diagnoses could be considered correct.

### Image Analysis

The efficacy of the system in quickly identifying changing or suspicious lesions is further enhanced by use of automatic image analysis. On the server side of the web interface, algorithms are able to generate 3D stereoscopic images of each lesion and compute various metrics important for diagnosing skin conditions. The “ABCs” of mole detection—asymmetry, border, color, diameter, elevation and overall evolution—are the gold standards for non-invasive diagnosis of melanoma. The server is capable of estimating all parameters needed to monitor these standard “ABC” variables. The image analysis addresses each query as listed below.

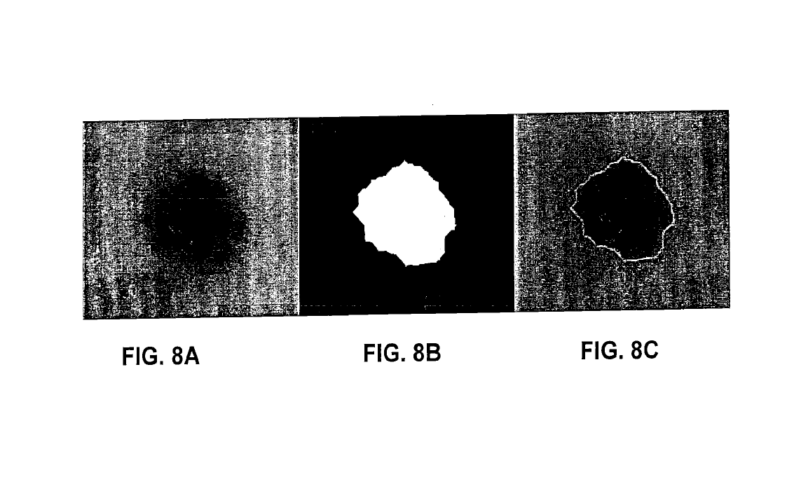

Asymmetry: the server first converts the image into a grey scale representation. A threshold is then determined to separate the abnormality from the background skin. FIG. 8A shows an image of a skin abnormality; FIG. 8B is the thresholded image for the same abnormality. The analysis software then draws a border along the largest isolated object, identifying the abnormality, and outputs this boundary on top of the original full color image (FIG. 8C). The center of this boundary is located, and distances between this center and the boundary are taken for the entire circumference. Values 180° apart are then compared and a fit value associated with the total difference is assessed. This value can be tracked over time to determine if an abnormality is becoming more asymmetric. The equation used to compute this difference is given by:

$${R}_{asymmetry} = {\sum }_{1}^{Total Pixels / 2} \vert \sqrt{{( {x}_{center} - {x}_{( i )} )}^{2} + {( {y}_{center} - {y}_{( i )} )}^{2}} - \sqrt{{( {x}_{center} - {x}_{( i + [ Total Pixels / 2 ] )} )}^{2} + {( {y}_{center} - {y}_{( i + [ Total Pixels / 2 ] )} )}^{2}}$$

This measurement will only be computed for nevi and circular abnormalities.

Border: A circle function can be fit to the boundary visualized in the asymmetry analysis. In order to track border changes, a computation is made to determine how well this imposed circle fits to the boundary. If the lesion becomes less circular in border behavior, this value increases. The equation used is as follows:

$${R}_{circular} = {\sum }_{1}^{Total Pixels} \vert \sqrt{{( {x}_{center} - {x}_{( i )} )}^{2} + {( {y}_{center} - {y}_{( i )} )}^{2}} - Radius$$

This measurement will only be computed for nevi and circular abnormalities.

Color: Due to the standardized LED polarized lighting, obtained images have consistent coloring with minimal glare. The abnormality is isolated from the backdrop of skin, and a histogram is computed based on colors found only within this region. The average color intensity and standard deviation are then found. If a color change occurs, the histogram will reflect the shift.

Diameter: Once the server traces a border around an abnormality, a circle function can be fit to approximate the region. This circle's diameter can then be computed and tracked over time.

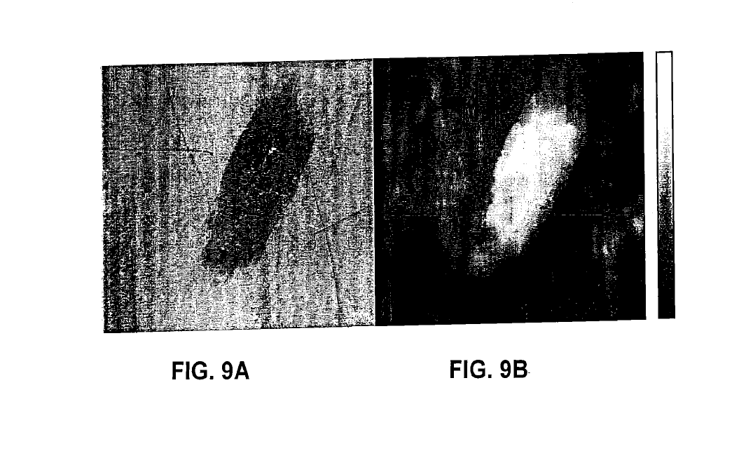

Elevation: Each pair of stereoscopic images is combined into a single height map for providing elevation information. Elevation values would then be tracked over time. FIG. 9A shows an image of a lesion having variations in height; FIG. 9B shows a height map for the same lesion. The light/dark scale accompanying FIG. 9B indicates that light areas are relatively higher regions and dark areas are relatively low regions.

Evolution: Due to the design of the interface and analysis, change over time is easily tracked for all of the previous metrics. The Doctor Portal clearly displays this information in graphical form, easily identifying significant changes and rates of change.

Other Lesions: Though the image analysis was originally focused on detecting various nevi characteristics, the interface has proven helpful in tracking a variety of different skin conditions. The analysis software fits a boundary to approximately 90% of all lesions imaged, and can compute the area of these abnormalities. A change in area would be diagnostically important for broader lesions. Color and elevation can also be tracked for these non-nevus conditions. Using these metrics broadens the system's applicability in the monitoring, diagnosis and treatment of skin abnormalities.

### CONCLUSION

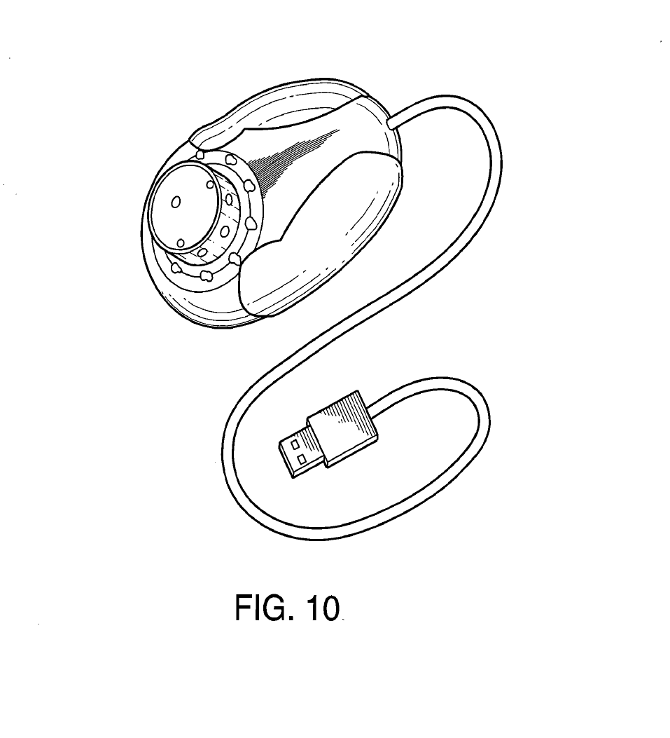

As described above, the 3Derm system is capable of capturing stereoscopic 3D images, and is also able to bring teledermatology to the patient. The system may be advantageously used in many situations where a low-cost, durable teledermatology solution is desired. The dermatoscope is a compact, ergonomic device (see FIG. 10) that may be used with a wide variety of computing equipment (desktop and laptop computers, mobile devices, etc.).

The capability to consult a dermatologist from the field may reduce the likelihood of a suspicious lesion being ignored or a benign lesion being exposed to unnecessary surgery. Patients in areas with limited numbers of dermatologists may also benefit from the ability to have suspicious abnormalities seen by a specialist.

The system provides patients' primary care physicians and dermatologists a portable, reliable and user-friendly option to identify, catalogue and monitor suspicious skin abnormalities. Its ease of use makes it an attractive option to keep track of moles and other skin abnormalities that may otherwise go unmonitored. By using this remote monitoring system, patients will be reassured that any changes in their condition will be quickly noticed and responded to. This will improve patient-doctor interactions by increasing their frequency and reducing the cost and time commitment. By making it easier to monitor skin abnormalities, the system will increase patient awareness of skin health and improve early cancer detection.

Besides skin imaging, the 3Derm system including a handheld, stereoscopic, low-power imaging microscope may have numerous other applications. Like other dermatoscopes, the 3Derm dermatoscope may be used for hair follicle examinations. More generally, biological imaging may be performed to produce large databases of 3D animal and plant images.

A system embodying the disclosure may also be used for material and textile inspections to improve quality control in manufacturing environments.

Crime investigators may also use the 3D dermatoscope device to image important pieces of evidence for documentation. The 3D capabilities could be especially useful to add a level of detail otherwise difficult to perceive with a standard imager.

While the disclosure has been described in terms of specific embodiments, it is evident in view of the foregoing description that numerous alternatives, modifications and variations will be apparent to those skilled in the art. Accordingly, the disclosure is intended to encompass all such alternatives, modifications and variations which fall within the scope and spirit of the disclosure and the following claims.

What is claimed is:

1. A system comprising:
an imaging device for imaging a skin abnormality of a patient, including
a camera; and
a controller for controlling linear motion of the camera over a predetermined distance so that the camera records a plurality of images from locations separated by said distance, thereby obtaining a stereoscopic image of the skin abnormality;
a data path for transmission of image data to a computing device; and
a web interface for linking the computing device to a web site providing access to storage of the image data.

2. A system according to claim 1, wherein the imaging device further includes
a track for mounting the camera thereon, and
a servo motor, connected to the camera and to the controller, for causing said linear motion along the track.

3. A system according to claim 1, wherein the camera includes LED illumination and a first polarizing filter.

4. A system according to claim 3, wherein the imaging device includes a second polarizing filter with polarization orthogonal to that of the first polarizing filter, thereby providing cross-polarized illumination of the skin abnormality.

5. A system according to claim 1, wherein the imaging device is configured as a handheld unit, and the camera and data path respectively are characterized as a plug-and-play webcam and a USB connection.

6. A system according to claim 1, wherein the web interface provides
a patient portal for entering patient information and information regarding the patient's skin abnormality; and
a doctor portal for accessing patient medical history and for entering clinical data regarding the patient's skin abnormality.

7. A system according to claim 6, wherein the web interface provides direct electronic communication between a patient and a doctor.

8. A system according to claim 6, wherein the web interface, via the doctor portal, has a link to a de-identified file sharing system, and has an encrypted link to a database storing patient health information (PHI), said encrypted link and database being HIPAA compliant.

9. A system according to claim 1, further comprising a server configured to receive and analyze the image data, generate 3D stereoscopic images of the skin abnormality, and compute metrics for clinical evaluation of the skin abnormality.

10. A system according to claim 9, wherein the server is configured to analyze elevation information relating to the skin abnormality, and generate a height map for the skin abnormality.

11. A system according to claim 9, further comprising an online database storing patient information including medical history and data regarding the skin abnormality, the database being accessible to a user via the web interface.

12. A system according to claim 9, wherein the server is configured to analyze a grey scale representation of an image of a skin abnormality with respect to a threshold value, thereby generating a thresholded image of the skin abnormality.

13. A system according to claim 9, wherein the server is configured to analyze an image of a skin abnormality to distinguish the skin abnormality from adjacent background skin, and to generate a boundary for the image of the skin abnormality.

14. A system according to claim 9, wherein said metrics include asymmetry, border, color, diameter, elevation and evolution of the skin abnormality.

15. A system according to claim 14, wherein the web interface provides a doctor portal for accessing patient medical history, entering clinical data regarding the patient's skin abnormality, and displaying information regarding evolution of the skin abnormality in graphical form.
